(19) United States
(12) Patent Application Publication
(10) Pub. No.: US 2002/0142476 A1
(43) Pub. Date: Oct. 3, 2002

(54) Method for dry-weight basis quantitative analysis of carbohydrates utilizing proton NMR

(76) Inventor: Douglas Wayne Lowman, Telford, TN (US)

Correspondence Address: Mark L. Davis, P.O. Box 9293, Gray, TN, 37615-9293, US

(21) Appl. No.: 09/782,761

(22) Filed: Feb. 13, 2001

(51) Int. Cl. G01N 33/10

(52) U.S. Cl. 436/94; 436/173

(57) ABSTRACT

A method for quantifying, on a dry-weight basis, an amount of carbohydrate, and preferably, (1→3)-β-D-glucan, contained in a sample. The method includes the steps of subjecting the carbohydrate-containing sample material to proton NMR analysis to produce a sample spectrum and quantifying the amount of carbohydrates present in the sample using an internal assay after compensating for the weight of solvent, such as water, present in the glucan-containing sample.

### BACKGROUND OF THE INVENTION

1. Field of the Invention:

The present invention relates to a method for quantitative determination of carbohydrates using proton nuclear magnetic resonance spectroscopy (NMR). Particularly, the present invention relates to an improved method for the quantitative determination, on a dry-weight basis, of an amount of carbohydrates in a complex matrix using proton NMR. More particularly, the present invention relates to a method for the quantitative determination, on a dry-weight basis, of an amount of (13)-p-D-glucan using proton NMR.

2. Background of the Invention:

Carbohydrates and sugars play an important role in today's society. They are included in the production of many food stuffs, such as cereals and sweetened drinks. Recently carbohydrates and sugars and derivatives thereof are finding use in the production of natural food supplements as well as pharmacological and dermatological medicants.

Carbohydrates are compounds of carbon, hydrogen and oxygen that contain the saccharide unit or its reaction product. Carbohydrates and their derivatives which can be utilized in the present invention include monosaccharides, such as fructose and glucose, disaccharides, such as sucrose, maltose, cellobiose and lactose; and polysaccharides, such as, starch and cellulose.

Starch is a mixture of linear (amylose) and branched (amylopectin) naturally occurring polymers having the D-glucopyranosyl repeat unit (glucose). It is the principle component of most plant seeds, tubes and roots and is produced commercially from corn, wheat, rice, tapioca, potato and other sources. Most commercial starch is produced from corn which is relatively inexpensive and abundant. Starch, as a polyhydroxy compound, may undergo many reactions characteristic of alcohols including esterification and etherification. For example, by reacting with metal hydroxides and alkylene oxides, various hydroxy alkyl starch derivatives such as hydroxyethyl and hydroxypropyl starches can be obtained. Cationic starches may be prepared from a starch slurry reacted with tertiary and quaternary alkyl amino compounds. The products are generally characterized as functional amine modified starches. Examples of cationically modified corn starch are CATO 31 and 237 available from National Starch and Chemical Company.

Cellulose is the primary framework of most of plants. For industrial purposes, cellulose is derived mainly from cotton linters or wood pulp by either mechanical or chemical processes. Cellulose esters such as cellulose formate, acetate, propionate, butyrate, valerate, caproate, heptylate, caprate, laurate, myristate and palmitate are obtained by reaction with organic acids, anhydrides or acid chlorides. Cellulose ethers are derived from the reaction of cellulose with alkylating agents such as chloroacetic acid and alkylene oxides under basic conditions. The cellulose ethers include, but are not limited to, anionic sodium carboxymethylcellulose (CMC) and nonionic hydroxyethylcellulose (HEC) and HEC modified with a long chain alkyl group, i.e. HMHEC(Hydrophobically Modified HEC). Cellulose ethers are available from Aqualon, under the trade name of Natrosol and Natrosol Plus.

Qualitative methods for detection of sugars include classical methods such as Fehling's solution for reducing sugars as well as paper and thin layer chromatography. Older quantitative methods include using hydrometry, polarimetry and spectrophotometry based on enzymic and color reactions to give the amounts of reducing and non-reducing sugars present. Separation and quantitation of each sugar present is possible by the derivatisation of the sugars followed by gas liquid chromatography or high performance liquid chromatography.

Recently, the polysaccharide (13)-p-D-glucan has greatly increased its importance in the nutraceutical industry in view of its reported health benefits. The glucan has been reported to provide inmmunobiological, hypocholesterolemic and hypoglycemic effects in humans and animals. These naturally occurring glucans, which can be isolated from plant cell walls, pathogens, such as bacteria, and fungi, comprise a class of drugs known as biological response modifiers. For example, U.S. Pat. No. 5,980,918 discloses a topical burn ointment containing P-D-glucan derived from plant materials such as oats, barley wheat or other cereal grains. Accordingly, isolation, characterization and assay of the glucan is critical to producing a viable glucan product while knowledge of the (13)-p-D-glucan purity is critical for optimum evaluation of the efficacy of the glucan in health-related applications.

Until now such characterization required several days and was very labor intensive. For example, it has been reported by N. R. Di Luzio, D. L. Williams, R. B. McNamee, B. F. Edwards, and Akio Kitahama in “Comparative tumor-inhibitory and anti-bacterial activity of soluble and particulate glucan”, Int. J. Cancer (1979), 24, 773-779, that particulate glucan recovery from Saccharomyces cerevisiae yeast can be accomplished by dispersing the glucan in 3% NaOH and heating to a boil for 4 hours. The material is cooled overnight and the supernatant is discarded. The NaOH digestion is repeated three times. The residue is then acidified with concentrated HCl plus 3% HCI and placed in a boiling water bath for 4 hours. The material is cooled overnight and the supernatant is discarded. The residue was further digested with 3% HCl at 100 ° C. for 4 hours. The 3% HCI digestion is repeated twice. The residue is washed three times with distilled water at 20 ° C. and twice with distilled water at 100 ° C. Ethanol is added to the residue and allowed to stand for a minimum of 24 hours for maximum extraction. The alcohol supernatant is aspirated from the residue and discarded. The alcohol extraction procedure is repeated until the alcohol supernatant was colorless. The alcohol is removed by washing the residue four times with hot water. The resulting particulate glucan is then collected by centrifugation.

A modification of the Di Luzio et al. isolation procedure above has been reported by D. L. Williams, R. B. McNamee, E. L. Jones, H. A. Pretus, H. E. Ensley, I. W. Browder and N. R. Di Luzio in “A method for solubilization of a (1→3)-β-D-glucan isolated from Saccharomyces cerevisiae”, Carbohydrate Research (1991), 219, 203-213. In this modified procedure, particulate glucan is recovered from Saccharomyces cerevisiae yeast by dispersing the glucan in 0.75 M (3%) NaOH and heating the solution to a boil. The material is cooled overnight and the supernatant is discarded. The NaOH digestion is repeated twice. The residue is then treated with 2.45 M HC1 and heated to a boil. The material is cooled overnight and the supernatant is discarded. The HC1 digestion is repeated twice using 1.75 M and 0.94 M HC1, respectively. Water is added to the residue which is then heated to a boil. The material is cooled overnight and the supernatant is discarded. The water wash is repeated until the residue becomes white and flocculent. The residue is then treated with ethanol and heated to a boil. The material is cooled overnight and the supernatant is discarded. The ethanol wash is repeated several times until the supernatant becomes colorless. Water is then added to the residue and heated to a boil. The material is cooled overnight and the supernatant is discarded. The water washing is repeated until the supernatant becomes colorless. The particulate glucan is then filtered, frozen and lyophilized to dryness. The process results in about a 2% yield by weight of the glucan on a solvent-free basis. Typical standard deviation for the determination of isolated glucan by these extraction methods is better than 3% (Muller, A.; Mayberry, W.; Acuff, R.; Thedford, S.; Browder, W.; Williams, D.; “Lipid content of microparticulate (→3)-β-D-Glucan isolated from Saccharomyces cerevisiae,Microbios, 1994, 79, 253-261).

Another modification of the isolation procedure of Williams et al (1991) above has been reported by A. Muller et al. (1994). Particulate glucan recovery from Saccharomyces cerevisiae yeast was accomplished using a process similar to that described by Williams et al (1991) with the following modifications: the particulate glucan was extracted with 1% (v/v) HCl in ethanol followed by 1% (v/v) NaOH in ethanol. The material is then washed extensively to remove the ethanol. The particulate glucan is lyophilized to dryness.

U.S. Pat. No. 5,811,542 discloses a process for producing soluble glucans which involves a sequence of acid and alkaline treatments. The process involves suspending glucan particles in an organic acid solution under conditions sufficient to dissolve the acid-soluble glucan portion. The acid-soluble glucans are then separated from the solution and then treated with NaOH to bring the pH to the range of 7 to 14. The slurry is then resuspended in hot alkali having a concentration and temperature sufficient to solubilize the glucan polymers. The particulate residue is then removed from the mixture. The mixture is chilled and the particulate is recovered and then washed with water and then resuspended in a sodium hydroxide solution having a pH in the range of 10 to 13. The resulting solution contains soluble glucan.

U.S. Pat. No. 6,008,054 issued to Izawa on Dec. 28, 1999 discloses a method for measuring P-glucan by flow injection using calcofluor, a fluorescent compound that specifically binds to P-glucan. The method utilizes a gel filtration column in which the volume of the interstice outside of the gel particles is not larger than the column effluent volume within a certain time period and the column content volume is greater than 10 times as large as the sample injection volume.

U.S. Pat. No. 6,084,092 issued to Wakshull et al. on July 4, 2000 discloses a method for quantifying the amount of (1→3)-p-β-glucan or (1→3)-β-D-glucan-containing organisms in a test sample. The method includes contacting the sample with a glucan binding agent, such as glycosphingolipids, under conditions that are suitable for binding the (13)-β-D-glucan present in the test sample. The resultant complexes are then isolated using standard techniques, such as immunoprecipitation, which results in concentrated primary complexes that are then separated using filtration techniques. The amount of (1→3)-β-D-glucan is then determined by contacting the resulting primary complexes with an antibody fragment or enzymatic reaction such as by Limulus amebocyte lysate or Limulus lysate Factor G assay.

As can be seen from the above, characterization of the (1→3)-β-D-glucan is very time intensive and involves multiple steps to isolate and evaluate the (1→3)-β-D-glucan. Another disadvantage of the aforementioned methods is they do not quantify the glucan in a solvent-wet matrix on a dry-weight basis.

Accordingly, there is a need for a method for determining the content of (1 →3)-β-D-glucan, on a dry-weight basis, in less time.

### SUMMARY OF THE INVENTION

Surprisingly, a method for determining the content of carbohydrates and particularly the content of(1→)-β-D-glucan, on a dry-weight basis has been found utilizing proton NMR analysis. The method includes subjecting a sample of(1→3)-β-D-glucan-containing material to NMR analysis and quantifying the amount of (1→3)-β-D-glucan present in the sample.

An object of the present invention is to provide a method for quantifying carbohydrates in crudely isolated mixtures without having to separate the carbohydrate from the mixture and its solvents.

It is another object of the present invention to provide a method for determining the content of (1→3)-β-D-glucan, on a dry-weight basis, in a sample in less time than previously known.

It is another object of the present invention to provide a method for quantitative glucan assay, on a dry-weight basis without the time-consuming multi-step separation and lyophilization to dryness.

Another object of the present invention is to improve the accuracy and reproducibility for measuring the amount of (1→3)-β-D-glucan, on a dry-weight basis, present in a sample utilizing proton NMR analysis.

Yet another object of the present invention is to provide a method for quantifying an amount of (1→3)-β-D-glucan, on a dry-weight basis, in a sample having less variability attributed to solvent content in the matrix.

These and other objects and advantages of the present invention will become more apparent to those skilled in the art in view of the following description and the accompanying drawings. It is to be understood that the inventive concept is not to be considered limited to the constructions disclosed herein but instead by the scope of the appended claims.

### BRIEF DESCRIPTION OF THE DRAWINGS

FIG. 1 is the proton NMR spectrum of a blank sample containing the internal standard dimethyl terephthalate (DMT), the solvent perdeuterated dimethylsulfoxide (DMSO-d₆) and trifluoroacetic acid-d (TFA), with pertinent resonances labeled.

FIG. 2 is the proton NMR spectrum of a sample containing isolated (1→3)-β-D-glucan from the yeast Saccharomyces cerevisiae, the internal standard (DMT) and TFA in DMSO-d₆ as described in Example 1.

FIG. 3 is the proton NMR spectrum of a sample from a crude isolation of (13)-P-D-glucan from Nutrex 370 inactive yeast, also containing the internal standard (DMT) and TFA in DMSO-d₆ as described in Example 3.

### DETAILED DESCRIPTION OF THE INVENTION

In accordance with the present invention, the method for quantifying carbohydrates, on a dry-weight basis, in a sample includes the steps of subjecting the sample containing the carbohydrate material to NMR analysis to produce a sample spectrum, and quantifying the amount of carbohydrates on a dry weight basis, in lieu of the presence of solvents and water present in the sample. The method of the present invention is particularly useful in quantifying the amount of (1→3)-β-D-glucan, on a dry-weight basis, in a complex matrix.

In the method of the present invention the type of carbohydrate is not critical and includes monosaccharides, such as fructose and glucose, disaccharides, such as sucrose, maltose, cellobiose and lactose; and polysaccharides, such as, starch and cellulose and preferably (1→3)-β-D-glucan.

Although the present invention will be hereinafter described with particularity as to (1→3)-β-D-glucan, one skilled in the art will readily understand the utility and applicability of the present method to the various forms of carbohydrates mentioned above.

The type of ( 1→3)-β-D-glucan which may be utilized in the method of the present invention is desirably soluble in a liquid material, preferably DMSO-d₆ or deuterated water. Such soluble glucan preparations can be prepared from insoluble materials as described in US Pat. No. 5,811,542. Moreover, the source of the glucan is not of particular importance and may be derived from a broad spectrum of glucan-containing organisms, such as from plant cell walls, pathogens, such as bacteria, fungi, and cereal grains. A particularly preferred source of (1→3)-β-D-glucan is obtained from Saccharomyces cerevisiae yeast cells.

A glucan product suitable for use in the present invention can have average particle size preferably of from about 1.0 microns or less, and more preferably of from about 0.20 microns or less. To obtain the particle size, the mixture containing the (1→3)-β-D-glucan may be ground down using a blender or ball mill, for example. One preferred grinding or particle size reduction method utilizes a blender having blunt blades, wherein the glucan mixture is blended for a sufficient amount of time, preferably several minutes, to completely grind the particles to the desired size without overheating the mixture. Another preferred grinding method comprises grinding the glucan mixture in a ball mill with 10 mm stainless steel grinding balls. This latter grinding method is particularly preferred when a particle size of about 0.20 microns or less is desired. Desirably, the water-soluble glucan has a molecular weight of between about 1,000 Daltons to about 100,000 Daltons with about 1000 to 30,000 Daltons being preferred.

The sample of (1→3)-β-D-glucan-containing material is dissolved in an appropriate solvent, such as DMSO-d₆ or deuterated water. The dissolved sample of ( 1→3)-β-D-glucan-containing material is subjected to proton NMR analysis. A proton NMR spectrometer and how it operates are well known to those skilled in the analytical art. Quantification of the (1→3)-β-D-glucan in the sample is accomplished by applying the internal standard assay technique to compare the area of a specific resonance of the glucan to the area of the resonance for the aromatic protons of DMT. Such techniques are known to those skilled in the art using a normal internal standard methodology.

In a preferred aspect of the present invention the method includes the steps of preparing a liquid sample of (1→3)-β-D-glucan-containing material by dissolving the (1→3)-β-D-glucan-containing material in DMSO-d₆ or deuterated water; adding to the dissolved (1→3)-β-D-glucan-containing material an amount of an internal standard, such as DMT, to make a liquid matrix; subjecting the liquid matrix containing the (1→3)-β-D-glucan to proton NMR analysis to produce a sample spectrum; and quantifying the amount of (1→3)-β-D-glucan present in the sample. The amount of internal standard added to the dissolved (1→3)-β-D-glucan-containing material should be sufficient to produce a measurable spectrum for the internal standard during NMR analysis.

The present invention is illustrated in greater detail by the specific examples presented below. It is to be understood that these examples are illustrative embodiments and are not intended to be limiting of the invention, but rather are to be construed broadly within the scope and content of the appended claims.

### EXAMPLE 1

Using the method described in Williams et al., 1991, the disclosure of which is incorporated herein by reference, glucan was isolated from Saccharomyces cerevisiae. The glucan (24.6 mg) and 16.0 mg of the internal standard, DMT, were weighed into a 3 dram glass vial and dissolved in 1 mL of DMSO-d₆. A blank sample containing 15.5 mg DMT in 1 mL of DMSO-d₆ in a 3 dram vial was also prepared. The glucan-containing vial and the blank vial were capped and placed on a hot plate at 80° C. and stirred using a magnetic stirring bar apparatus to dissolve the glucan, the internal standard, and any other DMSO-d6-soluble components present in the sample. After dissolution, the solutions were cooled and a few drops of trifluoroacetic acid-d (TFA, 99.8 % deuterated, Cambridge Isotope Laboratories) added to each vial and mixed with the solution. Addition of the TFA resulted in the shifting of the water resonance downfield to avoid any interference with the internal standard and glucan resonances of interest, as discussed in S. A. Ross and G. Lowe, “Downfield displacement of the NMR signal of water in deuterated dimethylsulfoxide by the addition of deuterated trifluoroacetic acid”, Tetrahedron Letters, 2000, 41, 3225-3227. The solutions were transferred to 5-mm OD NMR tubes and capped.

Proton NMR spectra of the solutions were obtained on a JEOL Model DELTA-400 NMR spectrometer operating at 399.78 MHz for proton observation and at 80 ° C. Field/frequency stabilization was accomplished using the deuterium of the solvent. NMR spectra were collected with 64 scans, 15 sec recycle delay, 32768 complex data points, and 15 ppm spectral window centered at 5.0 ppm. Spectra were processed with exponential apodization. Chemical shift referencing was accomplished relative to the residual proton resonance of DMSO-d₆ at 2.50 ppm.

The millimoles of water, as HOD, in the blank sample, mmolesHOD(Blank), were calculated to determine the amount of water added to the glucan-containing sample from the DMSO-d₆ and DMT using the following equation:

$$mmolesHOD ( Blank ) = \frac{4 * {W}_{DMT} {I}_{HOD}}{194.14 * {I}_{DMT}} \qquad Eqn 1$$

where W_{DMT}=weight of DMT, I_{HOD}=the integral area of the HOD proton resonance in rapid exchange with the deuterium of TFA, 194.14 =the molecular weight of the DMT, and I_{DMT}=the integral area of the singlet resonance from the 4 aromatic protons of DMT at 8.07 ppm.

The millimoles of water, as HOD, in the glucan-containing sample, mmolesHOD (Sample), were calculated using Equation 1.

The millimoles of glucan in the sample, mmolesGLUCAN, were calculated using the following equation:

$$mmolesGLUCAN = \frac{4 * {W}_{DMT} {I}_{glucan}}{194.14 * {I}_{DMT}} \qquad Eqn 2$$

where I_{glucan} is the integral area of the glucan anomeric proton resonance, H1, and other parameters are the same as in Equation 1. The structure for the glucan dimer repeat unit with protons numbered is shown below:

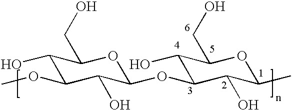

Proton chemical shift assignments for the numbered protons in the above structure were reported at 80 ° C. by H. S. Ensley, B. Tobias, H. A. Pretus, R. B. McNamee, E. L. Jones, I. W. Broder and D. L. Williams, “NMR spectral analysis of water-insoluble (1→3)-β-D-glucan isolated from Saccharomyces cerevisiae”, Carbohydrate Research 1994, 258, 307-311. The anomeric proton, Hi, resonates at 4.52 ppm. Proton H2 resonates at 3.28 ppm. Proton H3 resonates at 3.46 ppm. Protons H4 and H5 resonate at 3.25 ppm. The two H6 protons resonate at 3.7 ppm and 3.46 ppm.

Millimoles of HOD in the blank and the sample, mmolesHOD(Blank) and mmolesHOD(Sample), respectively, were converted to the weight of water in the blank and the glucan sample, wtH₂0(Blank) and wtH₂O (Sample), respectively. The weight contributed to the water resonance from the 3 exchangeable hydroxyl protons of the glucan, wt3OH(Glucan), were determined relative to the number of millimoles of glucan, mmolesGLUCAN, determined in Equation 2.

The weight of water in the water-wet sample, wtH₂O, is determined as follows:

wtH₂O=wtH₂O(Sample)−wtH₂O(Blank)−wt0OH(Glucan) The dry weight of the glucan sample, drywtGlucan, is calculated by difference as follows:

drywtGlucan=wtGlucan−wtH₂O Eqn 4

where wtGlucan is the weight of the glucan-containing sample added to the 3 dram vial.

The weight percent Glucan, (wt%Glucan), in the glucan sample on a dry-weight (i.e., solvent-free) basis is then calculated using a normal internal standard method as follows:

$$wt \% Glucan = \frac{400 * 162.15 * {W}_{DMT} * {I}_{Glucan}}{194.19 * drywtGlucan * {I}_{DMT}} \qquad Eqn 5$$

where 162.15 molecular weight of the glucosyl repeat unit and 400 represents the product of 4 for the 4 aromatic protons of DMT and 100 for the conversion to percent.

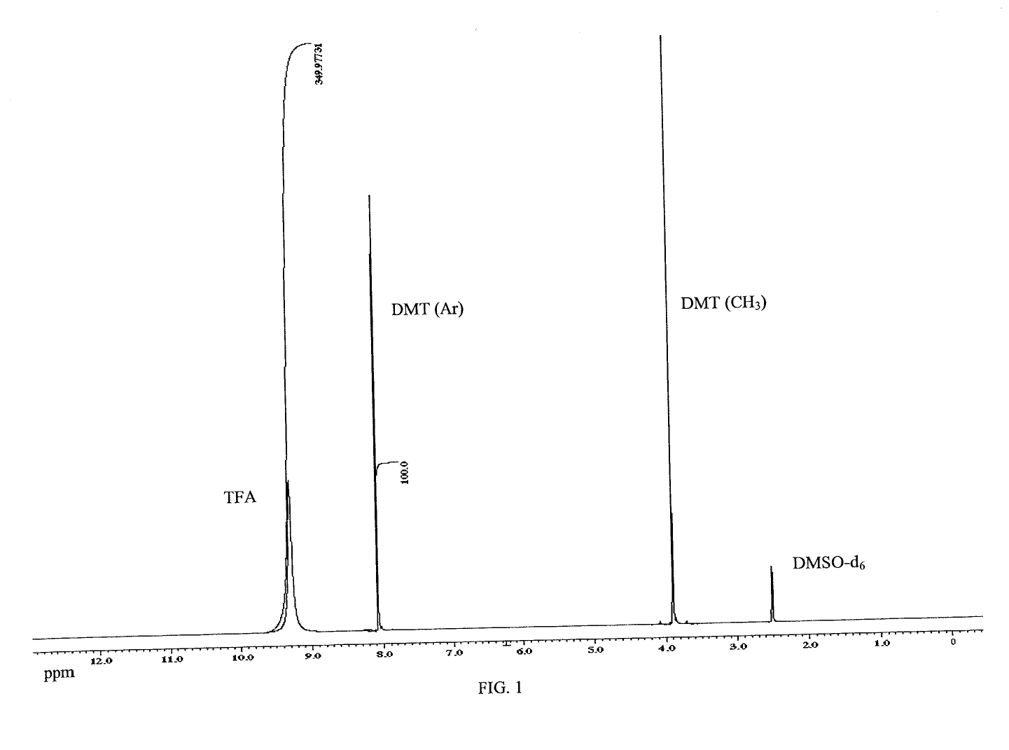

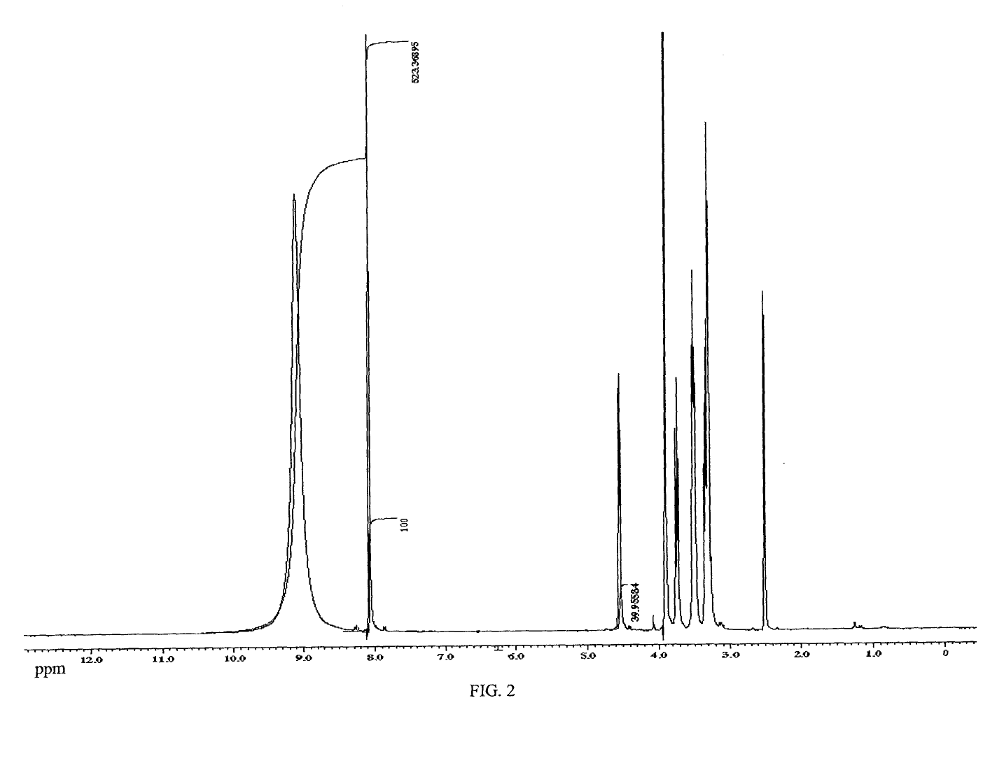

The proton NMR spectrum of the blank sample containing DMT and TFA in DMSO-d₆ is shown in FIG. 1. The proton NMR spectrum of the isolated glucan with DMT and TFA added is shown in FIG. 2. The isolated sample was calculated to contain 89.26 weight % glucan on a dry-weight basis. The glucan content of the sample was further analyzed using a standard total glucose assay after hydrolysis and was determined to be greater than about 90 weight %.

### EXAMPLE 2

Using the method described in Muller et al., 1994, the disclosure of which is incorporated herein by reference, the glucan was isolated from Saccharomyces cerevisiae. The glucan (24.84 mg) and 16.1 mg of the internal standard, DMT, were weighed into a 3 dram glass vial and dissolved in 1 mL of DMSO-d₆. A blank sample containing 15.5 mg DMT in 1 mL of DMSO-d₆ in a 3 dram vial was also prepared. Sample dissolution, data collection, and quantification were accomplished as described in Example 1. The isolated sample was calculated to contain 97.81 wt % glucan on a dry-weight basis. The glucan content of the sample was further analyzed using a standard total glucose assay after hydrolysis and was determined to be greater than about 98 weight %.

As can be seen from the above Examples, the method of the present invention provides a quick and accurate determination of glucan present in a glucan-containing sample.

### EXAMPLE 3

Glucan recovery from Nutrex 370 inactive yeast was accomplished by the following procedure: The yeast was dispersed in 500-mL distilled water and the pH adjusted to 13 by adding NaOH pellets. After heating the sample to reflux, 200-mL of absolute ethanol at 75 ° C. was added to reduce foaming. The material was refluxed at 85 ° C. After cooling the mixture, 300-mL more absolute ethanol was added aid in filtration. The filtered residue was washed with 100-mL distilled water and 300-mL ethanol. The resulting residue was recharged to a flask to which 500-mL distilled water was added. The pH was changed from 13 to 7.2 by adding sulfuric acid. After 100-mL ethanol was added, the material was held at reflux at 90 ° C. for 1 hour. The pH was changed to 0 by addition of more suliric acid and held at 90 ° C. for 1 hour. Ethanol (900-mL) was added to the cooled solution before filtering. The residue was washed with 500-mL 190 proof ethanol and filtered. To the residue was added 500-mL distilled water and sulfuric acid, then heated to reflux for 30 min at 92 ° C., then cooled and filtered after addition of 500-mL 190 proof ethanol. The residue was dried under vacuum at 100 ° C.

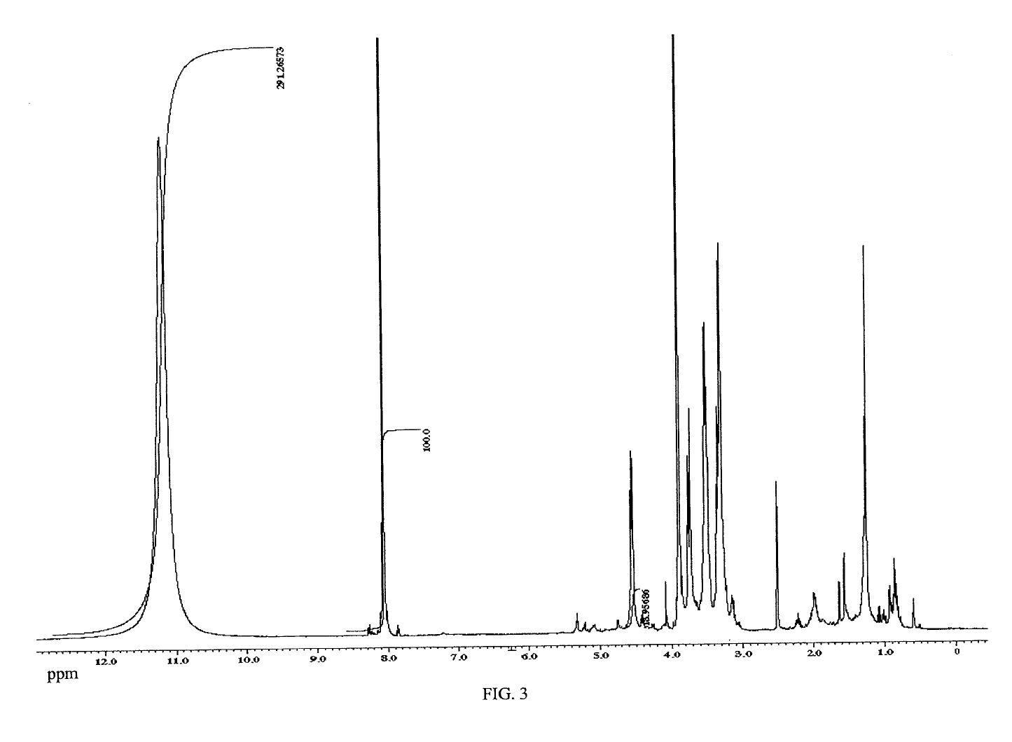

Approximately 25 mg of the glucan isolated from Nutrex 370 inactive yeast and about 15 mg of the internal standard, DMT, were weighed into a 3 dram glass vial and dissolved in 1 mL of DMSO- d₆. A blank sample containing 15.5 mg DMT in 1 mL of DMSO- d₆ in a 3 dram vial was also prepared. Sample dissolution, data collection, and quantification were accomplished as described in Example 1. The proton NMR spectrum of the crude isolation product from Nutrex 370 inactive yeast in the analysis mixture is shown in FIG. 3. The crude glucan-containing residue was found to contain 46.22 wt % glucan on a dry-weight basis.

As can be seen from the above examples, the method of the present invention provides several advantages over the heretofore described methods for measuring the amount of a carbohydrate, and particularly the amount of glucan, present in a sample. Surprisingly, the method of the present invention provides a rapid and accurate method by which the quantity of carbohydrates, dry-weight basis, can be determined in a wet sample without extensive separation and analytical process steps.

Having described the invention in detail, those skilled in the art will appreciate that modifications may be made to the various aspects of the invention without departing from the scope and spirit of the invention disclosed and described herein. It is, therefore, not intended that the scope of the invention be limited to the specific embodiments illustrated and described but rather it is intended that the scope of the present invention be determined by the appended claims and their equivalents. Moreover, all patents, patent applications, publications, and literature references presented herein are incorporated by reference in their entirety for any disclosure pertinent to the practice of this invention.

What is claimed is:

1. A method for measuring, on a dry-weight basis, the amount of a carbohydrate in a liquid matrix sample comprising:
a. subjecting said liquid sample carbohydrate-containing material to proton NMR analysis to produce a sample spectrum, and
b. quantifying the amount of carbohydrate present in the sample.

2. The method of claim 1 wherein said carbohydrate is selected from the group consisting of fructose, glucose, sucrose, maltose, cellobiose, lactose, starch and cellulose.

3. The method of claim 2 wherein said carbohydrate includes (1→3)-β-D-glucan.

4. The method of claim 1 further comprising dissolving said carbohydrate-containing material in a solvent to produce said liquid matrix prior to subjecting said liquid matrix to NMR analysis to produce a sample spectrum.

5. The method of claim 4 wherein said solvent is selected from the group consisting of DMSO-d₆ and deuterated water.

6. The method of claim 4 wherein said method further includes adding to said liquid matrix a sufficient amount of an internal standard to produce a measurable spectrum.

7. A method for measuring, on a dry-weight basis, the amount of (1→3)-β-D-glucan in a liquid matrix sample comprising:
a. subjecting said liquid sample of (1→3)-β-D-glucan-containing material to proton NMR analysis to produce a sample spectrum, and
b. quantifying the amount (1→3)-β-D-glucan present in the sample.

8. The method of claim 7 further comprising dissolving said (1→3)-β-D-glucan containing material in a solvent to produce said liquid matrix prior to subjecting said liquid matrix to NMR analysis to produce a sample spectrum.

9. The method of claim 8 wherein said solvent is selected from the group consisting of DMSO-d₆ and deuterated water.

10. A method for measuring, on a dry-weight basis, the amount of (1→3)-β-D-glucan in a liquid matrix comprising
a. dissolving said (1→3)-β-D-glucan-containing material in a solvent selected from the group consisting of DMSO-d₆ and deuterated water to produce a liquid matrix;
b. subjecting said liquid matrix to proton NMR analysis to produce a sample spectrum; and
c. quantifying the amount (1→3)-→-D-glucan present in the sample.

11. A method for measuring, on a dry-weight basis, the amount of (1→3)-β-D-glucan in a liquid matrix sample comprising:
a. preparing a liquid matrix sample of (1→3)-β-D-glucan-containing material by:
i. dissolving said (1→3)-β-D-glucan-containing material in a solvent selected from the group consisting of DMSO-d₆ and deuterated water; and
ii. adding to said dissolved (1→3)-β-D-glucan-containing material a sufficient amount of an internal standard to produce a measurable spectrum;
b. subjecting said liquid matrix to proton NMR analysis to produce a sample spectrum; and
c. quantifying the amount (1→3)-β-D-glucan present in the sample.

12. The method of claim 11 wherein said internal standard is DMT.

13. The method of claim 11 wherein said (1→3)-β-D-glucan has an average particle size of less than about 1.0 microns.

14. The method of claim 11 wherein said (1→3)-β-D-glucan has an average particle size of less than about 0.20 microns.

15. The method of claim 11 wherein said (1→3)-β-D-glucan has an average molecular weight of from about 1000 Daltons to about 100,000 Daltons.
